(19) United States
(12) Patent Application Publication
(10) Pub. No.: US 2011/0077727 A1
(43) Pub. Date: Mar. 31, 2011

(54) DNA Telomere Rejuvenation

(76) Inventor: Clyde Norman Shealy, Fair Groove, MO (US)

(21) Appl. No.: 12/503,848

(22) Filed: Sep. 28, 2009

(51) Int. Cl. A61N 1/02

(52) U.S. Cl. 607/152

(57) ABSTRACT

A copper screen placed between two one inch foam pads and covered with crushed sapphire crystal is connected to an activated Tesla coil. This leads to increased telomere length in normal white blood cells and can be used to increase the reproductive capacity of cells and to delay the onset of cellular senescence.

### FIELD OF THE INVENTION

This invention relates to methods and devices for extending the telomeres and therefore the regenerative capacity of cells. The invention, therefore, relates to the fields of chemistry and medical therapeutic and diagnostic technology.

### BACKGROUND OF THE INVENTION

A copper tubing pyramid or a copper screen is placed between two one inch foam pads. The copper screen is covered with crushed quartz or sapphire crystal. Either device is connected to a Tesla coil.

When the Tesla coil is activated, a field of 50 to 75 decibels at 54 to 78 GigaHz is created for total body immersion in these human DNA frequencies. In 6 individuals, I measured DNA telomeres at baseline and 3 months later after they had used the device 30 to 60 minutes daily at least 5 days each week. Ordinarily telomeres shrink 1% each year. In 4 of the 6 individuals, telomeres lengthened 1% and in the other 2, they did not shorten. This has tremendous potential for health and longevity. In three of the individuals who continued to use the invention, telomeres increased an average of 5.6%, instead of the expected 2% decrease.

### DESCRIPTION OF THE RELATED ART

Normal human somatic cells (i.e., fibroblasts, endothelial, and epithelial cells) display a finite replicative capacity of 50-100 population doublings characterized by a cessation of proliferation in spite of the presence of growth factors. This cessation of replication in vitro is variously referred to as cellular senescence or cellular aging. See, Goldstein, 1990, Science 249:1129, and Hayflick and Moorehead, 1961, Exp. Cell Res. 25:585. The replicative life span of cells is inversely proportional to the in vivo age of the donor, so cellular senescence is believed to play an important role in aging in vivo. See Martin et. al., 1979, Lab. Invest. 23:86, and Schneider and Mitsui, 1976, Proc. Natl. Acad. Sci. U.S.A. 73:3584.

Cellular immortalization (the acquisition of unlimited replicative capacity) may be thought of as an abnormal escape from cellular senescence. See Shay et al., 1991, Exp. Cell Res. 196:33. Normal human somatic cells appear to be mortal and to have limited longevity. In contrast, germline and malignant tumor cells appear to be relatively immortal and to have indefinite proliferative potential. Human cells cultured in vitro appear to require the aid of transforming viral oncoproteins to become immortal, and even then, the frequency of immortalization is 10.sup.−6 to 10.sup.−7. See Shay and Wright, 1989, Exp. Cell Res. 184:109. Cells obtained from murine sources immortalize at a relatively high frequency without the aid of transforming oncoproteins. A variety of hypotheses have been advanced over the years to explain the causes of cellular senescence.

Shay et al., 1992, Experimental Gerontology 27:477, and Shay et al., 1991, Exp. Cell Res. 196:33, describe a two-stage model for human cell mortality to explain the ability of Simian Virus 40 (SV40) T-antigen to immortalize human cells. The mortality stage 1 mechanism (M1) is a mechanism by which cells cease to proliferate after a certain number of population doublings, and the biological molecules that carry out this mechanism appear to be the target of certain tumor virus proteins. An independent mortality stage 2 mechanism (M2) produces crisis in cells that have bypassed M1, with crisis being typified by severe chromosomal abnormalities and ultimately cell death. The M2 mechanism thus prevents tumor viruses from directly immortalizing human cells. The papers noted above describe experiments in which T-antigen expression was driven by a mouse mammary tumor virus promoter to cause reversible immortalization of cells. SV40 T-antigen extends the replicative life span of human fibroblasts by an additional 40-60%. The M1 mechanism is overcome by T-antigen, perhaps by binding to various cellular proteins or by inducing new activities to repress the M1 mortality mechanism. The M2 mechanism then causes cessation of proliferation, even though the M1 mechanism is blocked. Immortality is achieved only when the M2 mortality mechanism is also disrupted. The M2 mechanism appears to cause a dominant repression of the immortal phenotype, because hybrids between mortal and immortal human cells are generally mortal. See Pereira-Smith and Smith, 1983, Science 221:964-966. As described above, such hybrids between murine cells immortalize at a much higher frequency, so it is relatively easy to obtain immortal hybrids between murine to lymphocyte and myeloma cells to obtain immortal antibody-producing hybridomas, for instance. However, such hybrids are not immortal when produced with human cells.

The finite replicative capacity of cells may reflect the work of a “clock” linked to DNA synthesis at the telomeres (ends) of chromosomes. Thus, Harley et al., 1990, Nature 345:458, state that the amount and length of telomeric DNA in human fibroblasts decreases as a function of serial passage during aging in vitro. Harley, 1991, Mutation Research 256:271, describes that telomeres of human somatic cells act as a mitotic clock, shortening with age both in vitro and in vivo in a replication dependent manner and proposes a hypothesis for human cell aging and transformation in which telomeres and telomerase, a ribonucleoprotein polymerase involved in telomeric DNA synthesis, play a causal role in cell senescence and cancer.

PCT patent publication No. 93/23572 describes therapeutic and diagnostic methods relating to telomerase and telomere length. The publication describes oligonucleotide reagents that either reduce loss of telomere length during passage of cells in vitro or increase telomere length of immortal cells in vitro.

There remains a need for a device that can increase telomere length in mortal cells and methods for extending the regenerative or reproductive capacity of mortal cells and hybrids between mortal and immortal human cells. The present invention meets these and other needs, as described below, first in summary fashion and then in detail with examples.

### SUMMARY OF THE INVENTION

A 39 inch wide one inch piece of foam, 78 inches long, has inside it a 36 inch copper screen, 76 inches long, with a two foot copper wire welded to one corner. One pound of crushed quartz or sapphire crystal is spread over the screen.

An identical piece of one inch foam is glued to the bottom piece so that the screen is enclosed between the two pieces of foam.

The foam is rounded at the 4 corners and covered completely with a sturdy plain fabric suitable for a mattress cover with the copper wire coming out a small button hole opening in one corner.

The copper wire welded to the copper screen extends out a small button hole opening in one corner of the cover. This wire is connected to the Tesla coil for operation.

### BRIEF DESCRIPTION OF THE DRAWING

FIG. 1 depicts a perspective view of an embodiment of the mat described herein with a cross-sectional view of one corner of the mat.

### DETAILED DESCRIPTION OF THE INVENTION

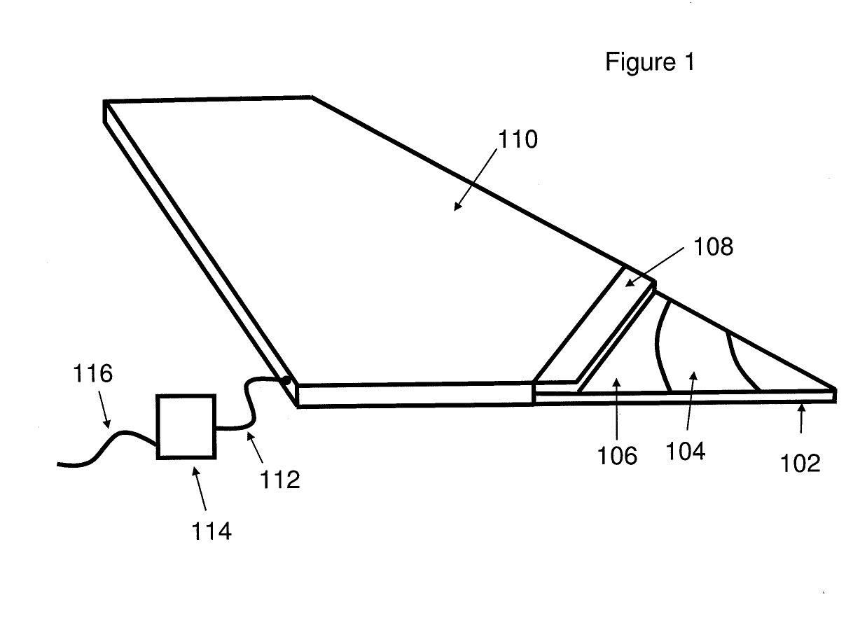

Referring now to FIG. 1, an embodiment of the mat of the present invention is depicted. In this embodiment of the mat, the bottom of the mat is formed by foam pad 102. In the corner of the mat which has been removed for clarity it can be seen that copper screen 104 is disposed on foam pad 102. Crushed crystal material 106 is disposed on the copper screen 104. A second foam pad 108 is disposed on top of the crystal and adhered to the bottom foam pad 102 along the adjacent edges. A cover 110 is disposed over the foam pads, typically with a zipper for ease of removing the cover for cleaning or as otherwise necessary.

A copper wire 112 is electrically connected to copper screen 104 and extends out from between foam pads 102 and 108, and through a hole provided in cover 110. Copper wire 112 is electrically connected to a Tesla coil 114. Coil 114 may be electrically connected to a power source by conductors 116.

In one embodiment, the RejuvaMatrix mat consists of a two inch thick mat, in the center of which is a copper screening similar to standard window screen, on which is scattered one pound of crushed sapphire crystal. A copper wire is attached to the copper screening and exits the center of the mat to be connected to a standard Tesla coil. The Tesla coil has an OUTPUT of 20,000 to 50,000 volts at frequency of approx. ½ megahertz. This output produces an electromagnetic field two feet high at 54 to 78 GHz, 75 decibels. The 54 to 78 GHz field is equal to the resonance frequency of human DNA.

The mat is covered with standard quilted mattress material.

The top and bottom polyfoam with copper screen and sapphire in the center are glued together with upholstery glue. The entire mat is enclosed with standard quilted mattress material with an opening for the copper wire leading to the Tesla coil.

1-3. (canceled)

4. A device for increasing the regenerative or reproductive capacity of normal cells having telomerase activity comprising:
a first foam pad and a second foam pad disposed adjacent to one another and secured to one another along the edges thereof;
a copper screen disposed between the first and second foam pads;
a quantity of particulate crystal material disposed evenly over the copper screen; and
a copper wire attached at a first end thereof to the copper screen and at a second end thereof to a Tesla coil.

5. A method for treating a patient comprising the steps of:
providing a mat comprising a first foam pad and a second foam pad disposed adjacent to one another and secured to one another along the edges thereof, with a copper screen disposed between the first and second foam pads, a quantity of particulate crystal material disposed evenly over the copper screen, and a copper wire attached at a first end thereof to the copper screen and a second end thereof to a Tesla coil;
disposing the patient on the mat in a sitting or lying position; and
energizing the Tesla coil.

6. The method of claim 5 wherein the patent is disposed on the mat while the Tesla coil is energized for between 30 and 60 minutes per day.

7. The method of claim 5 further comprising the step of immersing the body of the patient in a field of radiation between 54 and 78 Gigahertz.

8. The method of claim 7 wherein the field of radiation is between 50 and 75 decibels.
